(19) United States
(12) Patent Application Publication
(10) Pub. No.: US 2005/0124622 A1
(43) Pub. Date: Jun. 9, 2005

(54) Compounds and methods for the treatment of pain

(76) Inventors: Dean Gordon Brown, Wilmington, DE (US); Rebecca Ann Urbanek, Wilmington, DE (US); Wenhua Xiao, Montreal (CA); Thomas Michael Bare, West Chester, PA (US); Megan Murphy, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/168,755

(22) PCT Filed: Dec. 19, 2000
(86) PCT No.: PCT/SE00/02610

Related U.S. Application Data
(60) Provisional application No. 60/171,906, filed on Dec. 23, 1999.

(51) Int. Cl. A61K 31/503; C07D 487/04

(52) U.S. Cl. 514/248; 544/235

(57) ABSTRACT

2-substituted 7-chloro-4-hydroxy-1,2,5,10-tetrahydropyridazino[4,5-b]quinoline-1,10-dione compounds, pharmaceutical compositions and methods for the treatment of pain are disclosed.

### FIELD OF THE INVENTION

This invention relates to the treatment or prevention of pain or nociception.

### RELATED ART

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. This range of sensations, as well as the variation in perception of pain by different individuals, renders a precise definition of pain difficult, however, many individuals suffer with severe and continuous pain.

Pain that is caused by damage to neural structures is often manifest as a neural supersensitivity or hyperalgesia and is termed “neuropathic” pain. Pain can also be “caused” by the stimulation of nociceptive receptors and transmitted over intact neural pathways, such pain is termed “nociceptive” pain.

The level of stimulation at which pain becomes noted is referred to as the “pain threshold.” Analgesics are pharmaceutical agents which relieve pain by raising the pain threshold without a loss of consciousness. After administration of an analgesic drug a stimulus of greater intensity or longer duration is required before pain is experienced. In an individual suffering from hyperalgesia an analgesic drug may have an anti-hyperalgesic effect. In contrast to analgesics, agents such as local anaesthetics block transmission in peripheral nerve fibers thereby blocking awareness of pain. General anaesthetics, on the other hand, reduce the awareness of pain by producing a loss of consciousness.

Tachykinin antagonists have been reported to induce antinociception in animals, which is believed to be analogous to analgesia in man (Maggi et al, J. Auton. Pharmacol. (1993) 13, 23-93). In particular, non-peptide NK-1 receptor antagonists have been shown to produce such analgesia. For example, the NK-1 receptor antagonist RP 67,580 produced analgesia with potency comparable to that of morphine (Garret et al, Proc. Natl. Acad. Sci. USA (1993) 88, 10208-10212).

The opioid analgesics are a well-established class of analgesic agents with morphine-like actions. Synthetic and semi-synthetic opioid analgesics are derivatives of five chemical classes of compound: phenanthrenes; phenylheptylamines; phenylpiperidines; morphinans; and benzomorphans. Pharmacologically these compounds have diverse activities, thus some are strong agonists at the opioid receptors (e.g. morphine); others are moderate to mild agonists (e.g. codeine); still others exhibit mixed agonist-antagonist activity (e.g. nalbuphine); and yet others are partial agonists (e.g. nalorphine). Whilst an opioid partial agonist such as nalorphine, (the N-alkyl analogue of morphine) will antagonize the analgesic effects of morphine, when given alone it can be a potent analgesic in its own right.

Of all of the opioid analgesics, morphine remains the most widely used, but, in addition to its therapeutic properties, it has a number of drawbacks including respiratory depression, decreased gastrointestinal motility (resulting in constipation), nausea and vomiting. Tolerance and physical dependence also limit the clinical uses of opioid compounds.

Aspirin and other salicylate compounds are frequently used in treatment to interrupt amplification of the inflammatory process in rheumatoid diseases and arthritis and temporarily relieve the pain. Other drug compounds used for these purposes include phenylpropionic acid derivatives such as Ibuprofen and Naproxen, Sulindac, phenyl butazone, corticosteroids, antimalarials such as chloroquine and hydroxychloroquine sulfate, and fenemates (J. Hosp. Pharm., 36:622 (May 1979)). These compounds, however, are ineffective for neuropathic pain.

Available therapies for pain also have drawbacks. Some therapeutic agents require prolonged use before an effect is experienced by the patient. Other existing drugs have serious side effects in certain patients, and subjects must be carefully monitored to ensure that any side effects are not unduly threatening. Most existing drugs provide only temporary relief from pain and must be taken consistently on a daily or weekly basis. With disease progression the amount of medication needed to alleviate the pain often increases, thus increasing the potential for adverse side effects.

NMDA receptors are defined by the binding of N-methyl-D-aspartate (NMDA) comprise a receptor/ion channel complex with several different identified binding domains. NMDA itself is a molecule structurally similar to glutamate (Glu) which binds at the glutamate binding suite and is highly selective and potent in activating the NMDA receptor (Watkins (1987); Olney (1989)).

Many compounds are known that bind at the NMDA/Glu binding site (for example CPP, DCPP-ene, CGP 40116, CGP 37849, CGS 19755, NPC 12626, NPC 17742, D-AP5, D-AP7, CGP 39551, CGP43487, MDL-100,452, LY-274614, LY-233536, and LY233053). Other compounds, referred to as non-competitive NMDA antagonists, bind at other sites in the NMDA receptor complex (examples are phencyclidine, dizocilpine, ketamine, tiletamine, CNS 1102, dextromethorphan, memantine, kynurenic acid, CNQX, DNQX, 6,7-DCQX, 6,7-DCHQC, R(+)—HA-966, 7-chloro-kynurenic acid, 5,7-DCKA, 5-iodo-7-chloro-kynurenic acid, MDL-28,469, MDL-100,748, MDL-29,951, L-689,560, L-687,414, ACPC, ACPCM, ACPCE, arcaine, diethylenetriamine, 1,10-diaminodecane, 1,12-diaminododecane, ifenprodil, and SL-82.0715). These compounds have been extensively reviewed by Rogawski (1992) and Massieu et. al., (1993), and articles cited therein.

In addition to its physiological function, glutamate (Glu) can be neurotoxic. Glu neurotoxicity is referred to as “excitotoxicity” because the neurotoxic action of Glu, like its beneficial actions, is mediated by an excitatory process (Olney (1990); Choi (1992)). Normally, when Glu is released at a synaptic receptor, it binds only transiently and is then rapidly removed from the receptor by a process that transports it back into the cell. Under certain abnormal conditions, including stroke, epilepsy and CNS trauma, Glu uptake fails and Glu accumulates at the receptor resulting in a persistent excitation of electrochemical activity that leads to the death of neurons that have Glu receptors. Many neurons in the CNS have Glu receptors, so excitotoxicity can cause an enormous amount of CNS damage.

Acute excitotoxicity injury can occur as a result of ischemic events, hypoxic events, trauma to the brain or spinal cord, certain types of food poisoning which involve an excitotoxic poison such as domoic acid, and seizure-mediated neuronal degeneration, which can result from persistent epileptic seizure activity (status epilepticus). A large body of evidence has implicated the NMDA receptor as one receptor subtype through which Glu mediates a substantial amount of CNS injury, and it is well established that NMDA antagonists are effective in protecting CNS neurons against excitotoxic degeneration in these acute CNS injury syndromes (Choi (1988); Olney (1990)).

In addition to neuronal damage caused by acute insults, excessive activation of Glu receptors may also contribute to more gradual neurodegenerative processes leading to cell death in various chronic neurodegenerative diseases, including Alzheimer's disease, amyotrophic lateral sclerosis, AIDS dementia, Parkinson's disease and Huntington's disease (Olney (1990)). It is generally considered that NMDA antagonists may prove useful in the therapeutic management of such chronic diseases.

In the 1980's it was discovered that PCP (also known as “angel dust”) acts at a “PCP recognition site” within the ion channel of the NMDA Glu receptor. PCP acts as a non-competitive antagonist that blocks the flow of ions through the NMDA ion channel. More recently it has become evident that drugs which act at the PCP site as non-competitive NMDA antagonists are likely to have psychotomimetic side effects. Further, it is now recognized that certain competitive and non-competitive NMDA antagonists can cause similar pathomorphological effects in rat brain (Olney et. al., (1991); Hargreaves et. al., (1993)). Such compounds also have psychotomimetic effects in humans (Kristensen et. al., (1992); Herrling (1994); Grotta (1994)).

The glycine binding site of the NMDA receptor complex is distinguishable from the Glu and PCP binding sites. Also, it has recently been discovered that NMDA receptors occur as several subtypes which are characterized by differential properties of the glycine binding site of the receptor. Many compounds that bind at the NMDA receptor glycine site, useful for the treatment of stroke and neurodegenerative conditions, have been described in U.S. Pat. Nos. 5,604,227; 5,733,910; 5,599,814; 5,593,133; 5,744,471; 5,837,705 and 6,103,721.

### SUMMARY OF THE INVENTION

It has now been discovered that certain compounds which exhibit the property of binding to the NMDA receptor glycine site have utility for the amelioration of pain and particularly for the amelioration of neuropathic pain.

Therefore, in a first aspect the present invention provides compounds, or pharmaceutically-acceptable salts thereof, selected from:
7-chloro-4-hydroxy-2-(4-[1,3,4]oxadiazol-2-yl-benzyl)-1,2,5,10-tetrahydropyridazino[4,5-b]quinoline-1,10-dione;
7-chloro-4-hydroxy-2-[3-(1H-tetrazol-5-yl)-benzyl]-1,2,5,10-tetrahydropyridazino[4,5-b]quinoline-1,10-dione;
4-[(7-chloro-4-hydroxy-1,10-dioxo-2,5-dihydropyridazino[4,5-b]quinolin-2-yl)methyl]benzaldehyde;
N-(1-aza-2-{4-[(7-chloro-4-hydroxy-1,10-dioxo(2,5-dihydropyridazino[4,5-b]quinolin-2-yl))methyl]phenyl}vinyl)(tert-butoxy)carboxamide;
2-({4-[2-aza-2-(2-pyridylamino)vinyl]phenyl}methyl)-7-chloro-4-hydroxy-2,5-dihydropyridazino[4,5-b]quinoline-1,10-dione;
N-(-1-aza-2-{4-[(7-chloro-4-hydroxy-1,10-dioxo(2,5-dihydropyridazino[4,5-b]quinolin-2-yl))methyl]phenyl}vinyl)benzamide, and
2-{[4-(2-aza-2-{[(2,4,6-trimethylphenyl)sulfonyl]amino}vinyl)phenyl]methyl}-7-chloro-4-hydroxy-2,5-dihydropyridazino[4,5-b]quinoline-1,10-dione.

In another aspect the invention comprises a pharmaceutical composition comprising a pain-ameliorating effective amount of a compound, or a pharmaceutically-acceptable salt thereof, selected from:
7-chloro-4-hydroxy-2-(4-[1,3,4]oxadiazol-2-yl-benzyl)-1,2,5,10-tetrahydropyridazino[4,5-b]quinoline-1,10-dione;
7-chloro-4-hydroxy-2-[3-(1H-tetrazol-5-yl)-benzyl]-1,2,5,10-tetrahydropyridazino[4,5-b]quinoline-1,10-dione;
7-chloro-4-hydroxy-2-[3-hydroxy-5(hydroxymethyl)-2-methyl(4-pyridyl)]methyl-1,2,5,10-tetrahydropyridazino[4,5-b]quinoline-1,10-dione;
7-chloro-4-hydroxy-2-(tetrahydrofuran-2-ylmethyl)-1,2,5,10-tetrahydropyridazino[4,5-b]quinoline-1,10-dione;
7-chloro-4-hydroxy-2-[5-(4-methoxyphenyl)-[1,3,4]oxadiazol-2-ylmethyl]-1,2,5,10-tetrahydropyridazino[4,5-b]quinoline-1,10-dione;
7-chloro-4-hydroxy-2-[1-(4-methoxycarbonylphenyl)eth-1-yl)-1,2,5,10-tetrahydropyridazino[4,5-b]quinoline-1,10-dione;
4-[(7-chloro-4-hydroxy-1,10-dioxo-2,5-dihydropyridazino[4,5-b]quinolin-2-yl)methyl]benzaldehyde;
N-(1-aza-2-{4-[(7-chloro-4-hydroxy-1,10-dioxo(2,5-dihydropyridazino[4,5-b]quinolin-2-yl))methyl]phenyl}vinyl)(tert-butoxy)carboxamide;
2-({4-[2-aza-2-(2-pyridylamino)vinyl]phenyl}methyl)-7-chloro-4-hydroxy-2,5-dihydropyridazino[4,5-b]quinoline-1,10-dione;
N-(−1-aza-2-{4-[(7-chloro-4-hydroxy-1,10-dioxo(2,5-dihydropyridazino[4,5-b]quinolin-2-yl))methyl]phenyl}vinyl)benzamide, and
2-{[4-(2-aza-2-{[(2,4,6-trimethylphenyl)sulfonyl]amino}vinyl)phenyl]methyl}-7-chloro-4-hydroxy-2,5-dihydropyridazino[4,5-b]quinoline-1,10-dione;
   together with a pharmaceutically-acceptable excipient or diluent;

In a further aspect, the invention provides a method for the treatment of pain comprising administering a pain-ameliorating effective amount of a compound selected from:
7-chloro-4-hydroxy-2-(4-[1,3,4]oxadiazol-2-yl-benzyl)-1,2,5,10-tetrahydropyridazino[4,5-b]quinoline-1,10-dione;
7-chloro-4-hydroxy-2-[3-(1H-tetrazol-5-yl)-benzyl]-1,2,5,10-tetrahydropyridazino[4,5-b]quinoline-1,10-dione;
7-chloro-4-hydroxy-2-[3-hydroxy-5(hydroxymethyl)-2-methyl(4-pyridyl)]methyl-1,2,5,10-tetrahydropyridazino[4,5-b]quinoline-1,0-dione;
7-chloro-4-hydroxy-2-(tetrahydrofuran-2-ylmethyl)-1,2,5,10-tetrahydropyridazino[4,5-b]quinoline-1,10-dione;
7-chloro-4-hydroxy-2-[5-(4-methoxyphenyl)-[1,3,4]oxadiazol-2-ylmethyl]-1,2,5,10-tetrahydropyridazino[4,5-b]quinoline-1,10-dione;
7-chloro-4-hydroxy-2-[1-(4-methoxycarbonylphenyl)ethyl)-1,2,5,10-tetrahydropyridazino[4,5-b]quinoline-1,10-dione;
4-[(7-chloro-4-hydroxy-1,10-dioxo-2,5-dihydropyridazino[4,5-b]quinolin-2-yl)methyl]benzaldehyde;
N-(1-aza-2-{[4-[(7-chloro-4-hydroxy-1,10-dioxo(2,5-dihydropyridazino[4,5-b]quinolin-2-yl))methyl]phenyl}vinyl)(tert-butoxy)carboxamide;
2-({4-2-aza-2-(2-pyridylamino)vinyl]phenyl}methyl)-7-chloro-4-hydroxy-2,5-dihydropyridazino[4,5-b]quinoline-1,10-dione;
N-(-1-aza-2-{4-[(7-chloro-4-hydroxy-1,10-dioxo(2,5-dihydropyridazino[4,5-b]quinolin-2-yl))methyl]phenyl}vinyl)benzamide, and
2-{[4-(2-aza-2-{[(2,4,6-trimethylphenyl)sulfonyl]amino}vinyl)phenyl]methyl}-7-chloro-4-hydroxy-2,5-dihydropyridazino[4,5-b]quinoline-1,10-dione.

Other aspects of the invention are methods for making the compounds disclosed herein.

Yet other aspects of the invention are pharmaceutical compositions which contain a compound disclosed herein; the use of such compounds for the preparation of medicaments and pharmaceutical compositions, and a method comprising binding a compound of the invention to the NMDA receptor glycine site of a warm-blooded animal, such as a human being, so as to beneficially inhibit the activity of the NMDA receptor.

### DETAILED DESCRIPTION OF THE INVENTION

Compounds of the invention are those exemplified hereafter.

Suitable pharmaceutically-acceptable salts of compounds of the invention include acid addition salts such as methanesulphonate, fumarate, hydrochloride, hydrobroncide, citrate, tris(hydroxymethyl)aminomethane, maleate and salts formed with phosphoric and sulphuric acid. In other embodiments, suitable salts are base salts such as an alkali metal salts for example sodium, alkaline earth metal salts for example calcium or magnesium, organic amine salts for example triethylamine, morpholine, N-methylpiperidine, N-ethylpiperidine, procaine, dibenzylamine, choline, N,N-dibenzylethylamine or amino acids such as lysine.

To use a compound of the invention or a pharmaceutically-acceptable salt thereof for the therapeutic treatment, which may include prophylactic treatment, of pain in mammals, which may be humans, the compound can be formulated in accordance with standard pharmaceutical practice as a pharmaceutical composition.

Suitable pharmaceutical compositions that contain a compound of the invention may be administered in conventional ways, for example by oral, topical, parenteral, buccal, nasal, vaginal or rectal administration or by inhalation. For these purposes a compound of the invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions. A preferred route of administration is orally by tablet or capsule.

In addition to a compound of the present invention a pharmaceutical composition of this invention may also contain one or more other pharmacologically-active agents, or such pharmaceutical composition may be simultaneously or sequentially co-administered with one or more other pharmacologically-active agents.

Pharmaceutical compositions of this invention will normally be administered so that a pain-ameliorating effective daily dose is received by the subject. The daily dose may be given in divided doses as necessary, the precise amount of the compound received and the route of administration depending on the weight, age and sex of the patient being treated and on the particular disease condition being treated according to principles known in the art. A preferred dosage regime is once daily.

A further embodiment of the invention provides a pharmaceutical composition which contains a compound of the invention as defined herein or a pharmaceutically-acceptable salt thereof, in association with a pharmaceutically-acceptable additive such as an excipient or carrier.

A yet further embodiment of the invention provide the use of a compound of the invention, or a pharmaceutically-acceptable salt thereof, in the manufacture of a medicament useful for binding to the NMDA receptor glycine site in a warm-blooded animal such as a human being.

Still another embodiment of the invention provides a method of binding a compound of the invention to the NMDA receptor glycine site of a warm-blooded animal, such as a human being, in need of treatment for pain, which method comprises administering to said animal an effective amount of a compound of structural diagram I or a pharmaceutically-acceptable salt thereof.

Definitions:

Generally in the methods, processes and examples described herein:
concentrations were carried out by rotary evaporation in vacuo;
operations were carried out at ambient temperature, that is in the range 18-26° C. and under a nitrogen atmosphere;
column chromatography (by the flash procedure) was performed on Merck Kieselgel silica (Art. 9385) unless otherwise stated;
yields are given for illustration only and are not necessarily the maximum attainable;
the structure of the end-products of the formula I were generally confirmed by NMR and mass spectral techniques, proton magnetic resonance spectra were determined in DMSO-d₆ unless otherwise stated using a Varian Gemini 2000 spectrometer operating at a field strength of 300 MHz; chemical shifts are reported in parts per million downfield from tetramethylsilane as an internal standard (δ scale) and peak multiplicities are shown thus: s, singlet; bs, broad singlet; d, doublet; AB or dd, doublet of doublets; t, triplet, dt, double of triplets, m, multiplet; bm, broad multiplet; fast-atom bombardment (FAB) mass spectral data were obtained using a Platform spectrometer (supplied by Micromass) run in electrospray and, where appropriate, either positive ion data or negative ion data were collected, in this application, (M+H)⁺ is quoted; IR data was obtained with a Nicolet Avatar 360 FT-IR;
intermediates were not generally fully characterized and purity was in general assessed mass spectral (MS) or NMR analysis.

The following abbreviations and definitions when used, have the meanings, as follows:
CDCl₃ is deuterated chloroform;
CMC is 1-cyclohexyl-3-(2-morpholinoethyl)carbodiimide metho-p-toluenesulfonate;
DCM is dichloromethane;
DCU is dicyclohexyl urea;
DHC is 1,3-dicyclohexylcarbodiimide;
DMAP is 4-(dimethylamino)pyridine;
DMF is N,N-dimethylformamide;
DMSO is dimethylsulphoxide;
m/s is mass spectroscopy;
NMP is N-methylpyrrolidinone;
NMR is nuclear magnetic resonance;
p.o. is per os;
THF is tetrahydrofuran, and
t.i.d. is three times daily.

The examples and tests described herein are intended to illustrate but not limit the invention.

### EXAMPLES

### Example 1

### 7-Chloro-4-hydroxy-2-(4-[1,3,4]oxadiazol-2-yl-benzyl)-1,2,5,10-tetrahydropyridazino[4,5-b]quinoline-1,10-dione

### 2-p-Tolyl-[1,3,4]oxadiazole

Triethyl orthoformate (5 mL) was added to p-toluic hydrazide (2.0 g, 13.3 mmol) and the solution was heated to reflux for 6 hours. The volatiles were removed under reduced pressure, and the organic materials dissolved in ethyl acetate (100 mL). The organic layer was extracted with 10% aqueous potassium carbonate, washed with aqueous sodium chloride (saturated, 1×10 mL), and dried over Na₂SO₄. The organic layer was filtered and removed under reduced pressure to give the title compound as a tan solid (2.1 g, 99%). ¹H NMR (300 MHz, DMSO-d₆): δ 2.40 (s, 3H); 7.42 (d, 2H, J=8.1 Hz); 7.88 (d, 2H, J=8.1 Hz); 9.30 (s, 1H).

### 2-(4-Bromomethyl-phenyl)-[1,3,4]oxadiazole

2-p-Tolyl-[1,3,4]oxadiazole (1.44 g, 9.03 mmol) was slurried in carbon tetrachloride (40 mL) and to this was added N-bromosuccinimide (1.68 g, 9.45 mmol), and benzoyl peroxide (0.21 g, 0.90 mmol). The mixture was heated to reflux for 6 hours, and then cooled to room temperature. The solids were filtered off and discarded. The remaining material was dissolved into DCM (100 mL) and extracted with aqueous sodium bicarbonate (saturated, 2×50 mL). The organic layer was washed with aqueous sodium chloride (saturated), and dried over Na₂SO₄. The organic layer was filtered and removed under reduced pressure to give the title compound as a tan solid (1.70 g, 79%). (Contains about 5-10% of the α,α,-dibrominated material). ¹H NMR (300 MHz, DMSO-d₆): δ 4.79 (s, 2H), 7.67 (d, 2H, J=8.4 Hz); 8.02 (d, 2H, J=8.4 Hz); 9.32 (s, 1H).

### N′-(4-[1,3,4]Oxadiazol-2-yl-benzyl)-hydrazinecarboxylic Acid tert-butyl Ester

2-(4-Bromomethyl-phenyl)-[1,3,4]oxadiazole (0.56 g, 2.35 mmol) was dissolved in DMF (8 mL) and to this was added tert-butyl carbazate (0.28 g, 2.11 mmol) followed by N,N-diisopropylethylamine (0.33 g, 0.45 mL, 2.62 mmol). The solution was heated to 90° C. for 12 hours and then cooled to room temperature. The material was dissolved into ethyl acetate (150 mL) and extracted with water (3×40 mL) and aqueous sodium chloride (saturated, 1×15 mL). The organic layer was dried over Na₂SO₄, and evaporated to a thick oil. This residue was chromatographed (SiO₂, 1:1 hexanes:ethyl acetate) to give the title compound as a white solid (0.12 g, 20%). ¹H NMR (300 MHz, DMSO-d₆): δ 1.37 (s, 9H); 3.96 (d, 2H, J=3.9 Hz); 4.93 (m, 1H); 7.55 (d, 2H, J=8.4 Hz); 7.93 (d, 2H, J=8.4 Hz); 9.31 (s, 1H).

### Dimethyl 7-chloro-4-hydroxyquinoline-2,3-dicarboxylate

A stirred mixture of methyl 2-amino-4-chlorobenzoate (2.50 g, 13.5 mmol) and dimethyl acetylenedicarboxylate (2.05 g, 14.4 mmol) in tert-butanol (22 ml) was refluxed for 7 hours under a nitrogen atmosphere. After adding additional dimethyl acetylenedicarboxylate (1.16 g, 8.13 mmol) and refluxing another 2.5 hours, the reaction mixture was allowed to cool to room temperature and potassium tert-butoxide (1.56 g, 13.9 mmol) was added in one portion. A precipitate formed and the resulting mixture was refluxed for 1.5 hours. The mixture was cooled to room temperature and filtered to separate the solids, which were washed with tert-butanol and diethyl ether. The solids were dissolved in water and acidified with 1 N sulfuric acid to form a precipitate. The resulting mixture was extracted with DCM and the combined extracts were washed with brine and water, dried over MgSO₄, filtered and concentrated to give a green solid. Recrystallization of this material from methanol provided the title compound (1.15 g, 47%) as an off-white solid, mp 232-233° C.; MS (CI):296 (M+H). Analysis for C₁₃H₁₀ClNO₅: Calc'd: C, 52.81; H, 3.41; N, 4.74; Found: C, 52.75; H, 3.47; N, 4.69.

### 3-Carbomethoxy-7-chloro-4-hydroxyquinoline-2-carboxylic Acid

To a stirred suspension of dimethyl 7-chloro-4-hydroxyquinoline-2,3-dicarboxylate (1.0 g, 3.38 mmol) in water (20 mL) was added an aqueous solution of sodium hydroxide (0.27 g, 6.75 mmol). Upon addition, the suspension dissolved. The reaction mixture was warmed to 60° C. for 1 hour. After this time the reaction was cooled to room temperature and acidified with concentrated hydrochloric acid. The product was then extracted into diethyl ether and ethyl acetate. The organic extracts were dried over MgSO₄, filtered and concentrated in vacuo to provide the title compound as a solid (900 mg). This material was purified by recrystallization employing an ethyl acetate/hexane co-solvent system to provide the title compound (571 mg, 60%) as a white solid mp 296° C. (dec); MS (CI)=238 (M+H). Analysis for C₁₂H₈NO₅Cl.0.45 CH₃CO₂CH₂CH₃.0.10 H₂O: Calc'd: C, 51.30; H, 3.68; N 4.34, Found: C, 51.28; H, 3.62; N 3.97 ¹H NMR δ 8.22 (d, J=8.7 Hz, 1H), 7.92 (d, J=1.8 Hz, 1H), 7.28 (dd, J=8.7, 1.8 Hz, 1H), 3.90 (s, 3H).

### 3-Carbomethoxy-2-pyrrolidinocarbamide-7-chloro-4-hydroxyquinoline

To a suspension of 3-carbomethoxy-7-chloro-4-hydroxyquinoline-2-carboxylic acid (2.25 g, 8.0 mmol) in THF (20 mL) at ambient temperature under a N₂ atmosphere was added DHC (1.65 g, 8.0 mmol) and pyrrolidine (0.596 g, 8.4 mmol). The reaction was stirred room temperature for 15 hours after which time the by-product urea was removed via filtration. The desired product was purified via flash column chromatography employing 5% methanol in chloroform to provide the title compound (2.52 g, 94.3%) as a tan solid, mp=215° C.; MS (CI): 335 (M+H). 300 MHz ¹H NMR (DMSO-d₆): δ 8.12 (d, J=8.7 Hz, 1H), 7.60 (d, 1H, J=1.8 Hz), 7.47 (dd, 1H, J=8.8, 2.0 Hz), 3.69 (s, 3H), 3.40-3.49 (m, 2H), 3.27-3.33 (m, 2H), 1.80-1.96 (m, 4H).

### 7-Chloro-4-oxo-2-(pyrrolidinylcarbonyl)hydroquinoline-3-carboxylic Acid

To a suspension of 3-carbomethoxy-2-pyrrolidinocarbamide-7-chloro-4-hydroxy quinoline (2.52 g, 7.5 mmol) in de-ionized water (40 mL) was added dropwise a solution (20 mL) of an aqueous potassium hydroxide (882 mg, 15.75 mmol). Upon complete addition, the reaction was warmed to 60° C. After 3 hours, the reaction was filtered to remove a small amount of insoluble material. The filtrate was then acidified to pH=1 which yield a white precipitate. The solid was isolated by vacuum filtration, washed with water, and dried at 30° C. in vacuo for 16 hours. This provided the title compound (1.5 g, 64%) as a white solid, mp=225-8° C.; MS (CI): 321 (M+H). 300 MHz ¹H NMR (DMSO-d₆): δ 8.28 (d, J=8.8 Hz, 1H), 7.77 (s, 1H), 7.64 (d, 1H, J=8.7), 3.52-3.57 (m, 2H), 3.17-3.19 (m, 2H), 1.83-1.98 (m, 4H).

### N′-[7-Chloro-4-oxo-2-(pyrrolidine-1-carbonyl)-1,4-dihydroquinoline-3-carbonyl-N-(4-1,3,4]oxadiazol-2-yl-benzyl)-hydrazinecarboxylic Acid tert-butyl Ester

To a stirred slurry of 7-chloro-4-oxo-2-(pyrrolidinylcarbonyl)hydroquinoline-3-carboxylic acid (0.12 g, 0.39 mmol,) in THF (8 mL) was added CMC (0.19 g, 0.45 mmol) and the reaction was stirred for five minutes. To this mixture was added, via dropwise addition, a solution of N′-(4-[1,3,4]oxadiazol-2-yl-benzyl)-hydrazinecarboxylic acid tert-butyl ester (0.11 g, 0.45 mmol) and DMAP (ca. 0.003 g) in THF (2 mL). The mixture was then refluxed for 16 hours. The solution was filtered, washed with DCM (5 mL) and the insolubles collected. This insoluble material was then washed with water (50 mL) and then diethyl ether (20 mL). The remaining insoluble material was dried in vacuo to give the title compound as a white solid. (0.20 g, 89%). The material was used in the following reaction without further purification.

### 7-Chloro-4-hydroxy-2-(4-[1,3,4]oxadiazol-2-yl-benzyl)-1,2,5,10-tetrahydropyridazino[4,5-b]quinoline-1,10-dione

To a stirred solution of N′-[7-chloro-4-oxo-2-(pyrrolidine-1-carbonyl)-1,4-dihydroquinoline-3-carbonyl]-N′-(4-[1,3,4]oxadiazol-2-yl-benzyl)-hydrazinecarboxylic acid tert-butyl ester (0.20 g, 0.33 mmol) in THF (5 mL) was added methanesulfonic acid (0.80 mL) and the reaction was heated to 70° C. for 1 hour. The mixture was cooled to room temperature and stirred for 16 hours. The resultant orange solid was, collected by filtration and washed with THF. The material was sonicated for 20 minutes in diethyl ether/methyl alcohol (1:1, 5 mL) and then filtered. The material was dried to give the title compound as an off-white solid (0.56 g, 17%). ¹H NMR (300 MHz, DMSO-d₆): δ 2.31 (s, 3H); 5.18 (s, 2H); 7.44 (m, 3H); 7.83 (d, 2H, J=8.1 Hz); 8.04 (s, 1H); 8.14 (d, 1H, J=8.7 Hz); 10.38 (br s, 1H); 11.30 (s, 1H); 11.95 (br s, 1H).

### Example 2

### 7-Chloro-4-hydroxy-2-[3-(1H-tetrazol-5-yl)-benzyl]-1,2,5,10-tetrahydropyridazino[4.5-b]quinoline-1,10-dione.

### 3-(1H-Tetrazol-5-yl)-benzaldehyde

3-Cyanobenzaldehyde (2.0 g, 15.2 mmol) was dissolved in 2-methoxyethanol (8 mL) and to this was added sodium azide (0.91 g, 15.2 mmol) and lithium chloride (0.96 g, 22.8 mmol). The mixture was stirred and heated to reflux for 16 hours and then poured into a mixture of concentrated hydrochloric acid (3 mL) and crushed ice (˜30 g). The thick suspension was stirred until a fine pink solid had formed. The solids were filtered, washed with water and diethyl ether to give the title compound (1.21 g, 46%). ¹H NMR (300 MHz, DMSO-d₆): δ 7.86 (t, 1H, J=7.8 Hz); 8.13 (dd, 1H, J=1.5, 7.8 Hz); 8.37 (ddd, 1H, J=1.5, 1.5, 7.8 Hz); 8.56 (d, 1H, J=1.5 Hz); 10.13 (s, 1H).

### N′-[3-(1H-Tetrazol-5-yl)-benzylidene]-hydrazinecarboxylic Acid tert-butyl Ester

3-(1H-Tetrazol-5-yl)-benzaldehyde (1.21 g, 6.95 mmol) was dissolved in THF (40 mL) and to this was added tert-butyl carbazate (0.96 g, 7.30 mmol). The reaction was stirred at room temperature for 16 hours and the THF removed under reduced pressure. The residual solid was triturated with hexanes to give the title compound as an off-white solid (2.0 g, 100%). ¹H NMR (300 MHz, DMSO-d₆): δ 1.48 (s, 9H); 7.69 (t, 1H, J=7.8 Hz); 7.78 (d, 1H, J=7.3 Hz); 8.03 (d, 1H, J=7.8 Hz); 8.09 (s, 1H); 8.31 (s, 1H); 11.08 (br s, 1H).

### N′-[3-(1H-Tetrazol-5-yl)-benzyl]-hydrazinecarboxylic Acid tert-butyl Ester

N′-[3-(1H-Tetrazol-5-yl)-benzylidene]-hydrazinecarboxylic acid tert-butyl ester (0.50 g, 1.7 mmol) was added to a Parr shaker bottle and slurried in methyl alcohol (20 mL). To this was added 10% palladium-on-carbon (−100 mg) and the reaction was hydrogenated at 42 psi for 5 hours. The catalyst was filtered on diatomaceous earth, washed with methyl alcohol (2×100 mL), and the solvents were removed in vacuo to give a oil. The oil was dissolved in DCM (20 mL) and this was removed under reduced pressure to azeotrope away any residual methyl alcohol. The title compound was isolated as an oil (0.40 g, 60%). ¹H NMR (300 MHz, DMSO-d₆): δ 3.96 (br s, 2H); 4.94 (br s, 1H); 7.50 (m, 2H); 7.89 (m, 1H); 8.01 (s, 1H); 8.27 (br s, 1H). MS (−CI) m/z 289, 290.

### N′-[7-Chloro-4-oxo-2-(pyrrolidine-1-carbonyl)-1,4-dihydroguinoline-3-carbonyl]-N′-[3-(1H-tetrazol-5-yl)-benzyl]-hydrazinecarboxylic Acid tert-butyl Ester

To a stirred slurry of 7-chloro-4-oxo-2-(pyrrolidinylcarbonyl)hydroquinoline-3-carboxylic acid, Example 1, (0.43 g, 1.34 mmol) in THF (20 mL) was added CMC (0.68 g, 1.60 mmol) and the reaction was stirred for five minutes. To this mixture was added, via dropwise addition, a solution of N′-[3-(1H-tetrazol-5-yl)-benzyl]-hydrazinecarboxylic acid tert-butyl ester (0.42 g, 1.47 mmol) and DMAP (0.05 g, 0.41 mmol) in THF (5 mL), and the mixture was then refluxed for 16 hours. The solution was filtered and the insolubles washed with THF. The THF was removed under reduced pressure to give a sticky yellow oil. This material was chromatographed (SiO₂, 90/10) to give the title compound as a yellow solid (0.48 g, 60%). The material was used without further purification in the following reaction.

### 7-Chloro-4-hydroxy-2-[3-(1H-tetrazol-5-yl)-benzyl]-1,2,5,10-tetrahydropyridazino[4,5-b]quinoline-1,10-dione

To a stirred solution of N′-[7-chloro-4-oxo-2-(pyrrolidine-1-carbonyl)-1,4-dihydroquinoline-3-carbonyl]-N′-[3-(1H-tetrazol-5-yl)-benzyl]-hydrazinecarboxylic acid tert-butyl ester (0.48 g, 0.81 mmol) in THF (15 mL) was added methanesulfonic acid (1.9 mL) and the reaction was stirred at room temperature for 16 hours. The THF was removed under reduced pressure to yield an orange oil. To this was added diethyl ether (50 mL) and the reaction stirred for 10 minutes. The ether was carefully decanted to leave a thick orange oil. To this was added water (5 mL) and a fluffy yellow precipitate formed. The mixture was stirred for 10 minutes and then filtered. The solids were washed with water (10 mL) followed by diethyl ether (20 mL). The solids were collected and sonicated in methyl alcohol/diethyl ether (1:5, 5 mL) for 10 minutes. The solids were collected and dried under reduced pressure to give the title compound as a cream-colored solid (0.14 g, 41%,m.p. >275° C.). ¹H NMR (300 MHz, DMSO d₆): δ 5.22 (s, 2H); 7.44 (d, 1H, J=8.1 Hz); 7.56 (m, 2H); 7.95 (m, 2H); 8.03 (s, 1H); 8.15 (d, 1H, J=8.7 Hz), 11.97 (br s, 1H); 12.69 (br s, 1H).

### Example 3

### 7-Chloro-4-hydroxy-2-[3-hydroxy-5(hydroxymethyl)-2-methyl(4-pyridyl)]methyl-1,2,5,10-tetrahydropyridazino[4,5-b]quinoline-1,10-dione Methanesulfonate

### N-{1-Aza-2-[3-hydroxy-5(hydroxymethyl)-2-methyl(4-pyridyl)]vinyl}(tert-butoxy)-carboxamide Hydrochloride

To a stirred solution of tert-butylcarbazate (1.22 g, 9.82 mmol) in THF (40 mL) was added pyridoxal hydrochloride (2.00 g, 9.82 mmol). After 2 h at room temperature, the reaction was refluxed for 4 h, and the solvent was removed in vacuo. The resultant solid was triturated with hexanes and filtered to give the title compound as a white solid (3.00 g, 96%). ¹H NMR (300 MHz, DMSO-d₆): δ 1.50 (s, 9H); 2.59 (s, 3H); 4.70 (s, 2H); 8.19 (s, 1H); 8.51 (s, 1H); 12.06 (s, 1H); 12.72 (s, 1H).

### (tert-Butoxy)-N-[3-hydroxy-5(hydroxymethyl)-2-methyl(4-pyridylmethyl)]carboxamide

N-{1-Aza-2-[3-hydroxy-5(hydroxymethyl)-2-methyl(4-pyridyl)]vinyl}(tert-butoxy)-carboxamide hydrochloride (3.0 g, 9.46 mmol) was dissolved in methyl alcohol (90 mL) and placed in a Parr shaker bottle. To this was added 10% palladium-on-carbon (300 mg) and the reaction was hydrogenated at 40 psi for 24 h. The mixture was filtered through diatomaceous earth, which was then washed with methyl alcohol (3×100 mL). The combine filtrate and washes were concentrated in vacuo. The resulting solid residue was dissolved in DCM (400 mL) and the resulting solution washed with saturated sodium bicarbonate. The organic layer was washed with brine, dried over MgSO₄ and concentrated to give the title compound as a white solid (1.4 g, 52%). ¹H NMR (300 MHz, DMSO-d₆): δ 1.39 (s, 9H); 2.53 (s, 3H); 3.16 (s, 1H); 4.16 (s, 2H); 4.61 (s, 2H); 8.11 (s, 1H); 8.76 (br s, 1H).

### N-[(tert-Butoxy)carbonylamino][7-chloro-4-oxo-2-(pyrrolidinylcarbonyl)(3-hydroquinolyl]-N-[3-hydroxy-5(hydroxymethyl)-2-methyl(4-pyridylmethyl)]carboxamide

To a stirred slurry of 7-chloro-4-oxo-2-(pyrrolidinylcarbonyl)hydroquinoline-3-carboxylic acid, Example 1, (1.5 g, 4.94 mmol) in THF (100 mL) was added CMC (2.4 g, 5.68 mmol) and the reaction was stirred for five minutes. To this mixture was added via dropwise addition a solution of (tert-butoxy)-N-[3-hydroxy-5-hydroxymethyl-2-methyl-(4-pyridylmethyl)]carboxamide (1.4 g, 4.94 mmol) and DMAP (0.250 g, 2.04 mmol) in THF (20 mL), and the mixture was stirred at room temperature for 1 hour. The reaction mixture was filtered and the collected solids washed with DCM (2×150 mL). The combined filtrate and washes were washed with water (100 mL) and brine and then dried over MgSO₄ and filtered. The filtrate was concentrated to dryness to give the title compound as a yellow foam (1.7 g, 58%).

### 7-Chloro-4-hydroxy-2-[3-hydroxy-5(hydroxymethyl)-2-methyl(4-pyridyl)]methyl-1,2,5,10-tetrahydropyridazino[4,5-b]quinoline-1,10-dione Methanesulfonate

To a stirred solution of N-[(tert-butoxy)carbonylamino][7-chloro-4-oxo-2-(pyrrolidinylcarbonyl)(3-hydroquinolyl]-N-[3-hydroxy-5-hydroxymethyl-2-methyl-(4-pyridylmethyl)] carboxamide (1.72 g, 1.5 mmol) in THF (40 mL) was added methanesulfonic acid (5 mL) and the reaction was stirred overnight. The volatiles were removed in vacuo and the resultant oil was poured on to crushed ice. To this mixture was carefully added 10 N sodium hydroxide until a solid precipitate formed. The solution was filtered to give an orange solid. The resultant aqueous layer formed a fine precipitate which was collected by filtration, washed with diethyl ether and dried to give the title compound as an off-white powder (m.p. dec at 275° C.). ¹H NMR (300 MHz, DMSO-d₆): δ 2.30 (s, CH₃SO₃H); 2.39 (s, 3H); 4.84 (s, 2H); 5.21 (s, 2H); 7.45 (dd, 1H, J=2.1, 9.0 Hz); 8.03 (m, 2H); 8.13 (d, 1H, J=7.2 Hz). Calc'd. for C₁₈H₁₃ClN₄O₃.0.5 CH₃SO₃H.H₂O: C, 49.63; H, 3.87; N, 11.84. Found: C, 49.74; H, 3.84; N, 11.74.

### Example 4

### 7-Chloro-4-hydroxy-2-(tetrahydro-furan-2-ylmethyl)-1,2,5,10-tetrahydropyridazino[4,5-b]quinoline-1,10-dione

### 4-(2-Hydroxy-ethyl)-piperazine-1-carboxylic Acid tert-butyl Ester

1-(2-Hydroxy-ethyl)-piperazine (2.00 g, 1.88 mL, 15.3 mmol) was dissolved in DCM (80 mL) and to this was added di-tert-butyl dicarbonate (3.35 g, 15. 3 mmol). The reaction was stirred at room temperature for 72 hours, diluted with DCM (100 mL) and extracted with water (1×50 mL). The organic layer was then washed with aqueous sodium chloride (saturated, 1×50 mL) and dried over Na₂SO₄. The organic layer was collected and removed under reduced pressure to give the title compound as an oil (3.5 g, 99%). ¹H NMR (300 MHz, DMSO-d₆): δ 1.38 (s, 9H); 2.39 (m, 6H); 3.28 (t, 4H, J=5.1 Hz); 3.46 (dd, 2H J=6.0 Hz); 4.39 (s, 1H, J=6.0 Hz).

### 4-(2-Hydroxy-ethyl)-piperazine, Polystyrene Bound

4-(2-Hydroxy-ethyl)-piperazine-1-carboxylic acid tert-butyl ester (3.3 g, 14.3 mmol) was dissolved in THF (80 mL) and to this was added solid potassium tert-butoxide (1.57 g, 13.9 mmol). The mixture was stirred for 30 minutes at 0° C., and then at room temperature for 2 hours. The reaction mixture was then transferred to an Erlenmeyer flask which was charged with chloromethylated styrene/divinylbenzene copolymer resin (1% cross linked, 1 meq./gram, 2.8 g). The reaction flask was shaken for 48 hours, and the resin collected by filtration. The resin was washed with THF (2×50 mL), water (2×50 mL), water/DMF (1/1, 2×50 mL), DCM (2×50 mL) and ethyl acetate (2×50 mL). The resin was dried under reduced pressure to give polystyrene resin bound 4-(2-hydroxy-ethyl)-piperazine-1-carboxylic acid tert-butyl ester (3.1 g). IR (on resin) 1696 (s); 1492 (w); 1452 (w); 1242 (s); 1172 (s) cm⁻¹.

Polystyrene bound 4-(2-hydroxy-ethyl)-piperazine-1-carboxylic acid tert-butyl ester (3.1 g) was slurried in DCM (50 mL) and trifluoracetic acid (10 mL) at room temperature for 2 hours. The resin was collected by filtration and washed with 2 N ammoniated methanol (2×30 mL), DMF (1×40 mL), and DCM (3×30 mL). The resin was dried under reduced pressure to give the title compound (2.45 g). IR (on resin) 1601 (w); 1493 (w); 1452 (w).

### 7-Chloro-2-chlorocarbonyl-4-oxo-1,4-dihydroquinoline-3-carboxylic Acid Methyl Ester

3-Carbomethoxy-7-chloro-4-hydroxyquinoline-2-carboxylic acid (1.00 g, 3.55 mmol) was slurried in chloroform (25 mL, ethanol free) and to this was added thionyl chloride (˜2 mL). The mixture was heated to reflux for 6 hours, and then cooled to room temperature. The volatiles were removed under reduced pressure to give the title compound as a solid. This material was used in the following reaction without further purification. ¹H NMR (300 MHz, CDCl₃): δ 4.06 (s, 3H); 7.58 (dd, 1H, J=8.1 Hz); 8.06 (dd, 1H, J=1.8 Hz); 8.29 (d, 1H, J=8.1 Hz); 12.59 (s, 1H).

### 7-Chloro-2-[4-(2-hydroxy-ethyl)-piperazine-1-carbonyl]-4-oxo-1,4-dihydroquinoline-3-carboxylic Acid, Polystyrene Bound

Polystyrene resin-bound 4-(2-hydroxy-ethyl)-piperazine (2.45 g) was slurried in THF (50 mL) and to this was added 7-chloro-2-chlorocarbonyl-4-oxo-1,4-dihydroquinoline-3-carboxylic acid methyl ester (1.0 g, 3.55 mmol). The mixture was shaken for 16 hours, and the resin was collected by filtration. The resin was washed with THF (50 mL), DMF/water (1/1, 50 mL), DMF (50 mL), methyl alcohol (50 mL), and methanolic ammonia (0.5 N, 2×50 mL). The resin was dried under reduced pressure to give polystyrene resin bound 7-chloro-2-[4-(2-hydroxy-ethyl)-piperazine-1-carbonyl]4-oxo-1,4-dihydroquinoline-3-carboxylic acid methyl ester (2.10 g). IR (on resin) 1656 (w); 1601 (w); 1492 (w); 1467 (w); 1452 (w).

Polystyrene resin-bound 7-chloro-2-[4-(2-hydroxy-ethyl)-piperazine-1-carbonyl]-4-oxo-1,4-dihydroquinoline-3-carboxylic acid methyl ester (2.10 g) was added to a mixture of THF (10 mL) and water (4 mL). To this was added potassium hydroxide (0.35 g, 6.3 mmol) and the reaction stirred at reflux for 6 hours. The reaction was quenched by the addition of aqueous hydrochloric acid (3.10 mL, 2N) and the resin collected by filtration. The resin was washed with water (30 mL), DMF/water (1/1, 50 mL), DMF (50 mL), methyl alcohol (50 mL), and DCM (50 mL). The resin was then dried under reduced pressure to give the title compound (0.75 g). IR (on resin) 1666 (w); 1602 (w); 1582 (w); 1492 (w); 1452 (w).

### N-(1-Aza-2-(2-furanyl)vinyl)(tert-butoxy)carboxamide

To a stirred slurry of tert-butylcarbazate (131.5 g, 0.99 mol) in hexane (1000 mL) was added 2-furaldehyde (91.9 g, 0.95 mol). The slurry was refluxed for 2.5 h, and then cooled to room temperature. The resultant tan solid was filtered, dried and used as is in the next reaction (200 g, 99%). ¹H NMR (300 MHz, DMSO-d₆): δ 1.52 (s, 9H); 6.45 (dd, 1H, J=3.3, 1.2 Hz); 6.71 (d, 1H, J=3.3 Hz); 7.47 (d, 1H, J=1.2 Hz); 7.91 (s, 1H).

### (+/−)—N′-(Tetrahydro-furan-2-ylmethyl)-hydrazinecarboxylic Acid tert-butyl Ester

A solution of N′-(1-azz-2-(2-furanyl)vinyl(tert-butoxy)carboxamide (2.05 g, 9.80 mmol) was dissolved in 50 methyl alcohol (50 mL) and charged into a Parr shaker bottle. To this was added palladium on carbon (10%, 0.310 g) and the mixture shaken at 40 psi for 4 hours. The mixture was filtered on diatomaceous earth and solvent removed under reduced pressure to give the title compound as an oil. The material was used in the following reaction without further purification.

### (+/−)-N′-{7-Chloro-2-[4-(2-hydroxy-ethyl)-piperazine-1-carbonyl]-4-oxo-1,4-dihydroquinoline-3-carbonyl}-N′-(tetrahydro-furan-2-ylmethyl)-hydrazinecarboxylic Acid tert-butyl Ester, Polystyrene Bound

Polystyrene resin bound 7-chloro-2-[4-(2-hydroxy-ethyl)-piperazine-1-carbonyl]4 oxo-1,4-dihydroquinoline-3-carboxylic acid (0.50 g) was added to THF (30 mL) and to this was added CMC (1.0 g, 2.3 mmol). The reaction was allowed to shake for 15 minutes at which point the resin turned bright yellow. To this was added N′-(tetrahydro-furan-2-ylmethyl)-hydrazinecarboxylic acid tert-butyl ester (0.44 g, 2.05 mmol) and N,N-dimethylaminopyridine (0.10 g, 0.08 mmol) and the mixture was shaken for 16 hours. The resin was collected by filtration and washed with water (30 mL), DMF/water (1/1, 50 mL), DMF (30 mL), methyl alcohol (2×30 mL) and DCM (2×30 mL). The resin was dried under reduced pressure to give the title compound (0.40 g). IR (on resin) 1666 (w); 1602 (w); 1582 (w); 1492 (w); 1451 (w).

### (+/−)-7-Chloro-4-hydroxy-2-(tetrahydro-furan-2-ylmethyl)-1,2,5,10-tetrahydropyridazino[4,5-b]quinoline-1,10-dione

Polystyrene resin bound N′-{7-chloro-2-[4-(2-hydroxy-ethyl)-piperazine-1-carbonyl]4-oxo-1,4-dihydroquinoline-3-carbonyl}-N′-(tetrahydro-furan-2-ylmethyl)-hydrazinecarboxylic acid tert-butyl ester (0.40 g) was added to THF (15 mL) and to this was added methanesulfonic acid (1 mL). The mixture was shaken at room temperature for 24 hours. The resin was filtered and washed with THF. The solvent was evaporated and the residual oil diluted with of THF (3 mL). This mixture was poured into mixture of ice/water (80 mL) and stirred for 10 minutes. The resultant solid that formed was filtered and washed with diethyl ether (5 mL). The material was dried under reduced pressure to give the title compound as an off-white solid (0.01 g). ¹H NMR (300 MHz, DMSO-d₆): δ 1.65 (m, 1H); 1.85 (m, 3H); 3.61 (m, 1H); 3.75 (m, 1H); 3.87 (m, 1H); 3.95 (m, 1H); 4.20 (m, 1H); 7.42 (d, 1H, J=8.1 Hz); 8.02 (s, 1H); 8.14 (d, 1H, J=8.1 Hz); 12. 35 (br, s). MS (+CI) m/z 348/349.

### Example 5

### 7-Chloro-4-hydroxy-2-[5-(4-methoxy-phenyl)-[1,3,4]oxadiazol-2-ylmethyl]-1,2,5,5,10-tetrahydropyridazino[4,5-b]quinoline-1,10-dione

### 2-Chloromethyl-5-(4-methoxy-phenyl)-[1,3,4]oxadiazole

4-Methoxybenzoic hydrazide (0.70 g, 4.21 mmol) was dissolved in ethyl alcohol (10 mL) and to this was added 2-chloromethyl acetimidate hydrochloride (Schindler H., Helv. Chim. Acta 37 472-82 1954) (1.0 g, 6.36 mmol). The solution was stirred and heated to reflux for 2 hours. The mixture was dissolved in ethyl acetate (100 mL) and extracted with aqueous sodium bicarbonate (saturated, 1×20 mL), water (1×20 mL) and aqueous sodium chloride (saturated, 1×20 mL). The organic layer was dried over Na₂SO₄ and solvent removed under reduced pressure to give the title compound as a white solid. (0.83 g, 94%). ¹H NMR (300 MHz, DMSO-d₆): δ 3.86 (s, 3H); 5.12 (s, 2H); 7.16 (d, 2H, J=8.4 Hz); 7.92 (d, 2H, J=8.4 Hz).

### N′-[5-(4-Methoxy-phenyl)-[1,3,4]oxadiazol-2-ylmethyl]-hydrazinecarboxylic Acid tert-butyl Ester

2-Chloromethyl-5-(4-methoxy-phenyl)-[1,3,4]oxadiazole (0.83 g, 3.70 mmol) was dissolved in DMF (5 mL) and to this was added tert-butyl carbazate (1.22 g, 9.26 mmol) and N,N-di-iso-propylethylamine (0.71 g, 0.96 mL, 5.55 mmol). The solution was stirred and heated to 90° C. for 5 hours. The mixture was dissolved in ethyl acetate (150 mL) and extracted with water (3×15 mL), and brine (1×20 mL). The material was dried over Na₂SO₄ and the organic solvent removed under reduced pressure. The residual oil was triturated with hexanes/ethyl acetate (7:3, 10 mL) giving rise to a white precipitate. The precipitate was filtered and washed with small portions of hexanes/ethyl acetate (3×5 mL) giving the title compound as a white solid (0.75 g, 64%). ¹H NMR (300 MHz, DMSO-d₆): δ 1.34 (s, 9H); 3.85 (s, 3H); 4.08 (d, 2H, J=4.2 Hz); 5.27 (m, 1H); 7.15 (d, 2H, J=8.4 Hz); 7.93 (d, 2H, J=8.4 Hz); 8.39 (br s, 1H).

### N′-[7-Chloro-4-oxo-2-(pyrrolidine-1-carbonyl)-1,4-dihydroguinoline-3-carbonyl]-N′-[5-(4-methoxy-phenyl)-[1,3,4]oxadiazol-2-ylmethyl]-hydrazinecarboxylic Acid tert-butyl Ester

To a stirred slurry of 7-chloro-4-oxo-2-(pyrrolidinylcarbonyl)hydroquinoline-3-carboxylic acid, Example 1, (0.54 g, 1.66 mmol) in THF (10 mL) was added CMC (0.87 g, 2.07 mmol) and the reaction was stirred for five minutes. To this mixture was added, via dropwise addition, a solution of N′-[5-(4-methoxy-phenyl)-[1,3,4]oxadiazol-2-ylmethyl]-hydrazinecarboxylic acid tert-butyl ester (0.56 g, 1.75 mmol) and DMAP (0.01 g, 0.08 mmol) in THF (2 mL), and the mixture was then refluxed for 6 hours. The solution was filtered and the insolubles washed with THF. The THF was removed under reduced pressure to give a sticky yellow oil. The material was used without further purification in the following reaction (˜1 gram material isolated).

### 7-Chloro-4-hydroxy-2-[5-(4-methoxy-phenyl)-[1,3,4]oxadiazol-2-ylmethyl]-1,2,5,10-tetrahydropyridazino[4,5-b]quinoline-1,10-dione

To a stirred solution of N′-[7-chloro-4-oxo-2-(pyrrolidine-1-carbonyl)-1,4-dihydroquinoline-3-carbonyl]-N′-[5-(4-methoxy-phenyl)-[1,3,4]oxadiazol-2-ylmethyl]-hydrazinecarboxylic acid tert-butyl ester (0.30 g, 0.48 mmol) in THF (15 mL) was added methanesulfonic acid (1.0 mL) and the reaction was stirred at room temperature for 2 hours. The THF was removed under reduced pressure to yield an orange oil. To this was added diethyl ether (50 mL) and the reaction stirred for 10 minutes. The ether was carefully decanted to leave a thick orange oil. To this was added water (20 mL) and a fluffy yellow precipitate formed. The mixture was stirred for 10 minutes and then filtered. The solids were washed with water (10 mL) followed by diethyl ether (20 mL). The solids were collected and sonicated in methyl alcohol/diethyl ether (1:9, 5 mL) for 10 minutes. The solids were collected and dried under reduced pressure to give the title compound as a cream-colored solid (0.53 g, 25%). ¹H NMR (300 MHz, DMSO-d₆): δ 3.83 (s, 3H); 5.42 (s, 2H); 7.13 (d, 2H, J=8.7 Hz); 7.45 (d, 1H, J=8.7 Hz); 7.89 (d, 2H, J=8.7 Hz); 8.04 (s, 1H); 8.15 (d, 1H, J=8.7 Hz); 12.03 (br s, 1H); 12.89 (br s, 1H). MS (+CI) m/z 452/454.

### Example 6

### (+/−)-7-Chloro-4-hydroxy-2-[1-(4-methoxycarbonylphenyl)ethyl]-1,2,5,10-tetrahydropyridazino[4,5-b]quinoline-1,10-dione

### 4-[1-(tert-Butoxycarbonyl-hydrazino)-ethyl]-benzoic Acid Methyl Ester

To a solution of methyl 4-acetylbenzoate (5.20 g, 29.2 mmol) and tert-butylcarbazate (3.85 g, 29.2 mmol) in THF (70 mL) was added concentrated HCl (3 drops). Upon stirring 2 hours a solid formed. At that time the mixture was evaporated to dryness by rotary evaporation. The resulting solid was triturated with hexanes (3×50 mL) and dried in vacuo (500 mTorr, 30° C.) for 30 minutes to give the title compound as an off-white solid (8.36 g, 98%). ¹H NMR (300 MHz, DMSO-d₆): δ 1.49 (s, 9H); 2.22 (s, 3H); 3.34 (s, 1H); 3.86 (s, 3H); 7.87 (d, 2H, Jₒ=8.4 Hz); 7.97 (d, 2H, Jₒ=8.4 Hz); 9.97 (s, 1H).

### 4-[1-(tert-Butoxycarbonyl-hydrazino)-ethyl]-benzoic Acid Methyl Ester

To a suspension of 4-[1-(tert-butoxycarbonyl-hydrazino)-ethyl]benzoic acid methyl ester (8.35 g, 28.6 mmol) in ethanol (200 mL) was added 10% palladium on carbon (ca. 0.5 g) and the mixture was shaken under hydrogen at 50 psi on a Parr shaker for 3.5 hours. All the organic material dissolved during this time. The catalyst was removed by vacuum filtration through diatomaceous earth. The filtrate was evaporated by rotary evaporation. The residual solid was subjected to chromatography (silica gel, gradient of ethyl acetate/DCM beginning from 10/90 and ending at 20/80, respectively) to give the title compound as a white solid (6.41 g, 76%). ¹H NMR (300 MHz, DMSO-d₆): δ 1.18 (d, 3H, J=6.6 Hz); 1.35 (s, 9H); 3.84 (s, 3H); 4.16 (m, 1H); 4.71 (m, 1H); 7.48 (d, 2H, Jₒ=8.3 Hz); 7.89 (d, 2H, Jₒ=8.3 Hz); 8.15 (s, 1H).

### 4-(1-{N′-tert-Butoxycarbonyl-N-[7-chloro-4-hydroxy-2-(pyrrolidine-1-carbonyl)-quinoline-3-carboxyl]-hydrazino}-ethyl)-benzoic Acid Methyl Ester

To a stirred slurry of 7-chloro-4-oxo-2-(pyrrolidinylcarbonyl)hydroquinoline-3-carboxylic acid, Example 1, (2.15 g, 6.7 mmol) in THF (50 mL) was added CMC (5.72 g, 13.5 mmol) and the reaction was stirred for five minutes. To this mixture was added a solution of 4-[1-(tert-butoxycarbonyl-hydrazino)-ethyl]-benzoic acid methyl ester (1.97 g, 6.7 mmol) and DMAP (0.10 g, 0.84 mmol) in THF (10 mL), and the mixture was heated to reflux for 3 hours. The reaction mixture was allowed to cool, then the solids were filtered off and washed with DCM (2×50 mL). The combined filtrates were evaporated to dryness by rotary evaporation. The residual solid was subjected to chromatography (silica gel, 5/95 methanol/DCM) to give the title compound as an off-white solid (3.53 g, 71%). MS (CI) m/z 597/599.

### (+/−)-7-Chloro-4-hydroxy-2-[ethyl-1-(4-carbomethoxyphenyl)-1,2,5,10-tetrahydropyridazino[4,5-b]quinoline-1,10-dione

To a solution of 4-(1-{N′-tert-butoxycarbonyl-N-[7-chloro-4-hydroxy-2-(pyrrolidine-1-carbonyl)-quinoline-3-carboxyl]-hydrazino}-ethyl)-benzoic acid methyl ester (3.45 g, 5.78 mmol) in THF (50-mL) was added methanesulfonic acid (5 mL). After 18 hours the solvent was removed by rotary evaporation and the product was precipitated by addition of water (100 mL). The solid was collected by vacuum filtration and washed with water (2×50 mL) then diethyl ether (50 mL), and dried in vacuo (500 mTorr, 30° C.) for 1.5 hours. The solid was suspended in diethyl ether (175 mL) and methanol (25 mL) and sonicated for 20 minutes. The solid was collected by vacuum filtration and dried in vacuo (500 mTorr, 30° C.) for 18 hours. The resulting yellow solid (2.05 g, 83%) was found to be of insufficient purity by ¹H NMR. A portion of this material (1.73 g) was suspended in acetonitrile (10 mL) and water (10 mL) to which was added 20% choline hydroxide in water dropwise until all the solids dissolved, then purified by preparative HPLC (41.4 mm×25 cm C-18 Dynamax 60A column using a gradient 10-35% of acetonitrile in water with 0.1% TFA over 50 minutes with a flow rate of 20 ml/min, monitoring, 210 nM). Product was precipitated from the fractions containing title material by addition of 6M HCl to pH 3. The solid was collected by vacuum filtration, washed with water (2×50 ml) then diethyl ether (2×50 ml) and dried in vacuo (500 mTorr, 30° C.) for 18 hours to give the title compound as a yellow solid (1.23 g, 59%). ¹H NMR (300 MHz, DMSO-d₆): δ 1.69 (d, 3H, J=6.9 Hz); 3.84 (s, 3H); 6.26 (q, 1H, J=7.2 Hz); 7.38-7.50 (m, 3H); 7.92 (d, 2H, Jₒ=8.4 Hz); 8.03 (d, 1H, Jₘ=1.8 Hz); 8.15 (d, 1H, Jₒ=8.7 Hz); 11.94 (s, 1H); 12.57 (s, 1H). MS (CI) m/z 426/428. Calc'd. for C₂₁H₁₆ClN₃O₅.0.2 H₂O: C 58.74H 3.85 N 9.79. Found C 58.71-58.85H 3.89-3.90 N 9.93-9.95.

### Example 7

### 4-[(7-Chloro-4-hydroxy-1,10-dioxo-2.5-dihydropyridazino[4,5-b]quinolin-2-yl)methyl]benzaldehyde

### N-{1-Aza-2-[4-(diethoxymethyl)phenyl]vinyl}-(tert-butoxy)carboxamide

4-(Diethoxymethyl)phenyl carboxaldehyde (2.0 g, 10.0 mmol) was dissolved in methyl alcohol and to this was added tert-butyl carbazate (1.33 g, 10 mmol). The reaction was stirred overnight and used without further purification in the next reaction. Analysis: ¹H NMR (300 MHz, DMSO-d₆): δ 1.15 (t, 6H, J=6.9 Hz); 1.47 (s, 9H); 3.55 (m, 4H); 7.43 (d, 2H, J=8.1 Hz); 7.60 (d, 2H, J=8.1 Hz); 8.01 (br s, 1H); 10.93 (s, 1H).

### (tert-Butoxy)-N-{[4-((diethoxymethyl)phenyl)methyl]amino}Carboxamide

The methanolic solution from the previous step was added to a Parr shaker apparatus and charged with 10% palladium on carbon (0.300 g) and N-{1-aza-2-[4-(diethoxymethyl)phenyl]vinyl}-(tert-butoxy)carboxamide (2.50 g, 7.75 mmol) in methanol (40 mL) was hydrogenated (40 psi) at room temperature for 2 hours. The reaction was filtered through diatomaceous earth and the filtrate evaporated under reduced pressure to give the title compound as a gold oil (2.26 g, 70% for two steps). ¹H NMR (300 MHz, DMSO-d₆): δ 1.13 (t, 6H, J=6.9 Hz); 1.37 (s, 9H); 3.45 (m, 4H); 3.85 (s, 2H); 4.73 (br s, 1H); 7.32 (app s, 4H); 8.22 (br s, 1H).

### N-{[4-diethoxymethyl)phenyl]methyl}N-[(tert-butoxy)carbonylamino][7-chloro-4-oxo-2-(pyrrolidynlcarbonyl)(3-hydroquinolyl)]carboxamide

7-chloro-4-oxo-2-(pyrrolidinylcarbonyl)hydroquinoline-3-carboxylic acid, Example 1, (2.13 g, 6.66 mmol), was slurried in DCM (60 mL) and to this was added 1-(3-dimethylaminopropyl)-3-ethylcarbodiimide hydrochloride (7.20 g, 17.0 mmol). The reaction was stirred for five minutes at which point (tert-butoxy)-N-{[4-((diethoxymethyl)phenyl)methyl]amino}carboxamide (2.26 g, 6.99 mmol) and DMAP (0.19 g, 1.55 mmol) were added rapidly as a solution in DCM. The reaction was refluxed for three hours and then stirred at room temperature overnight. The reaction was further diluted with DCM (60 mL) and then extracted with water (30 mL), sodium bicarbonate (1×30 mL) and sodium chloride (20 mL). The organic layer was dried over Na₂SO₄ and concentrated to give the title compound as a yellow foam. The material was used without further purification.

### 4-[(7-chloro-4-hydroxy-1,10-dioxo-2,5-dihydropyridazino[4,5-b]quinolin-2-yl)methyl]benzaldehyde

A solution of N-{[4-diethoxymethyl)phenyl]methyl}N-[(tert-butoxy)carbonylamino] [7-chloro-4-oxo-2-(pyrrolidynlcarbonyl)(3-hydroquinolyl)]carboxamide (1.69 g, 2.71 mmol) was dissolved in THF (100 mL) and to this was added methanesulfonic acid (7 mL). The reaction was refluxed for one hour and then the volatiles removed in vacuo. To the residual oil was added diethyl ether (50 mL) and the mixture stirred for five minutes. The ether layer was decanted to leave a brown oil. To this oil was added water (50 mL) and the reaction stirred until a precipitate formed. The precipitate was collected by vacuum filtration, washed with diethyl ether, and dried in vacuo to give the title compound as a yellow solid (0.82 g, 80%). ¹H NMR (300 MHz, DMSO-d₆): δ 5.21 (s, 2H); 7.42 (d, 1H, J=8.7 Hz); 7.49 (d, 2H, J=8.1 Hz); 7.87 (d, 2H, J=8.1 Hz); 8.03 (s, 1H); 8.15 (d, 1H, J=8.7 Hz); 9.98 (s, 1H); 11.96 (s, 1H); 12.71 (s, 1H). MS (+CI) m/z 382/384.

### Examples 8-11

The compounds of Examples 8-11 were made by the following general procedure:

4-[(7-Chloro-4-hydroxy-1,10-dioxo-2,5-dihydropyridazino[4,5-b]quinolin-2-yl)methyl]benzaldehyde (0.1 g, 0.2 mmol) was slurried in methyl alcohol and to this was added the indicated hydrazine (0.2 mmol). The reaction mixture was refluxed for two hours, and cooled to room temperature. In most cases, a precipitate had formed which was filtered off, washed with diethyl ether and dried in vacuo to give the title compound. If a precipitate did not form, the methyl alcohol was removed in vacuo and the residual solid was sonicated in methyl alcohol/diethyl ether (1:1, 5 mL). This solid was collected by vacuum filtration, washed with diethyl ether and dried in vacuo to give the title compound.

### Example 8

### N-(1-Aza-2-{[4-[(7-chloro-4-hydroxy-1,10-dioxo(2,5-dihydropyridazino[4,5-b]quinolin-2-yl))methyl]phenyl}vinyl)(tert-butoxy)carboxamide

The title compound was prepared from tert-butyl carbazate as the starting hydrazine (8%). ¹H NMR (300 MHz, DMSO-d₆): δ 1.45 (s, 9H); 5.12 (s, 2H); 7.32 (d, 2H, J=8.1 Hz); 7.43 (d, 1H, J=8.7 Hz); 7.55 (d, 2H, J=8.1 Hz); 7.96 (s, 1H); 8.02 (s, 1H); 8.15 (d, 1H, J=8.7 Hz); 10.86 (br s, 1H); 11.92 (s, 1H); 12.66 (br s, 1H). MS (+CI) m/z 496/498.

### Example 9

### 2-{[4-[2-Aza-2-(2-pyridylamino)vinyl]phenyl}methyl)-7-chloro-4-hydroxy-2,5-dihydropyridazino[4,5-b]quinoline-1,10-dione

The title compound was prepared from 2-hydrazinopyridine as the starting hydrazine (48%). ¹H NMR (300 MHz, DMSO-d₆): δ 5.12 (s, 2H); 6.75 (t, 1H, J=6.3 Hz); 7.20 (d, 1H, J=8.1 Hz); 7.32 (d, 2H, J=8.1 Hz); 7.42 (d, 1H, J=8.7 Hz); 7.60 (d, 2H, J=8.7 Hz); 7.99 (s, 1H); 8.02 (s, 1H); 8.09 (d, 1H, J=4.8 Hz); 8.14 (d, 1H, J=8.7 Hz); 10.86 (br s, 1H); 11.94 (br s, 1H); 12.67 (br s, 1H). MS (+CI) m/z 473/475.

### Example 10

### N-((-1-aza-2-{4-[(7-chloro-4-hydroxy-1,10-dioxo(2,5-dihydropyridazino[4,5-b]quinolin-2-yl))methyl]phenyl}vinyl)benzamide

The title compound was prepared from benzhydrazide as the starting material (41%). ¹H NMR (300 MHz, DMSO-d₆): δ 5.15 (s, 2H); 7.38 (d, 2H, J=8.1 Hz); 7.42 (d, 1H, J=8.7 Hz); 7.55 (m, 3H); 7.68 (d, 2H, J=8.1 Hz); 7.90 (d, 2H, J=8.1 Hz); 8.03 (s, 1H); 8.15 (d, 1H, J=8.7 Hz); 8.43 (s, 1H); 11.83 (s, 1H); 11.95 (br s, 1H); 12.65 (br s, 1H). MS (+CI) m/z 500/502.

### Example 11

### 2-{[4-(2-Aza-2-{[(2,4,6-trimethylphenyl)sulfonyl]amino}vinyl)phenyl]methyl}-7-chloro-4-hydroxy-2,5-dihydropyridazino[4,5-b]quinoline-1,10-dione

The title compound was prepared from 2,3,6-trimethylsulfonyl hydrazide as the starting material (46%). ¹H NMR (300 MHz, DMSO-D₆): δ 2.22 (s, 3H); 2.62 (s, 6H); 5.10 (s, 2H); 7.02 (s, 2H); 7.28 (d, 2H, J=8.1 Hz); 7.43 (m, 3H); 7.87 (s, 1H); 8.02 (s, 1H); 8.14 (d, 1H, J=8.7 Hz); 11.55 (br s, 1H); 11.96 (br s, 1H); 12. 64 (br s, 1H).

Tests for Biological Function:

Test A: Inhibition of Binding of [³H]-MDL105,519:

Binding of compounds to the NMDA receptor glycine site may be assessed by measuring the ability of test compounds to inhibit the binding of tritiated MDL105,519 to brain membranes bearing the receptor.

Rat Brain Membranes: The rat brain membranes used in the experiments were obtained from Analytical Biological Services Inc., and were prepared substantially in accordance with the method of B. M. Baron et al., J. Pharmacol. Exp. Ther. 250, 162 (1989). Briefly, fresh brain tissue including cerebral cortex and hippocampus from male Sprague Dawley rats was homogenized in 0.32 M sucrose and centrifuged at low speed to separate cellular membranes from other cellular components. The membranes were then washed 3 times using deionized water, followed by treatment with 0.04% Triton X-100. Finally, membranes were washed six times in 50 mM Tris citrate buffer, pH 7.4, and frozen at −80° C. until use.

[³H]MDL105,519 (72 Ci/mmol) was purchased from Amersham. Cold MDL105,519 was purchased from Sigma/RBI. Binding assays were performed substantially in accordance with the protocol of B. M. Baron et al., J. Pharmacol. Exp. Ther. 279, 62 (1996), as follows. On the day of the experiment, brain membranes were thawed at room temperature and suspended in 50 mM tris acetate buffer, pH 7.4 (“TAB”). Seventy-five micro grams per milliliter protein (by using the BioRad dye) were used for competition binding. The experiments were carried out using 96-well plates. Membranes were incubated with 20 μL of compounds of various concentrations and 1.2 nM [³H]MDL105,519 for 30 minutes at room temperature in a total volume of 250 μL. Non specific binding was determined by using 100 μM of unlabeled MDL105,519. The unlabeled MDL105,519 and compounds were dissolved as 12.5 mM stock solutions in DMSO. Final DMSO concentration in each well was kept below 1%, which concentration was found not to alter the binding results. After incubation, unbound [³H]MDL105,519 was removed by filtration onto GF/B Unifilter plates using a Packard harvester. Filters were washed four times with ice cold TAB (total of 1.2 mL buffer). The plates were dried overnight at room temperature and bound radioactivity was measured on a Packard TopCount after the addition of 45 μL per well of the MICROSCINT O.

Human Brain Membranes: Human brain membranes were obtained from Analytical Biological Services Inc., and assays were performed as described for rat membranes.

Data analysis: Data was analyzed using a Microsoft Excel spreadsheet and GraphPad Prizm software and potency of compounds is expressed as the Ki (nM).

Test B: Formalin test:

The Formalin test is an assay that assesses the capacity of a compound to inhibit formalin-induced nociceptive behaviors in rats (D. Dubuisson, et al., Pain 4, 161-174 (1977); H. Wheeler-Aceto et al., Psychopharmacology 104, 3544 (1991); T. J. Coderre, et al., Pain 54, 43-50 (1993)). In the test, two distinctive phases of formalin-induced behaviors are observed. A first phase response, caused by acute nociception to the noxious chemical (formalin) injected into the paw, occurs between zero and five minutes. A quiescent period of 5 to 15 min post injection follows. After the quiescent period a second phase response, caused by sensitization of the central neurons in the dorsal horn, occurs after 15 minutes and lasts up to 60 minutes. Sensitization of the central neurons in the spine augments a noxious afferent input and causes a stronger pain barrage to be transmitted to the brain. Therefore, inhibition of the second phase response indicates a central mechanism of drug action.

The procedure for the formalin test may be performed as follows: male rats are placed in a plexiglass chamber and observed for 30-45 min. to observe their baseline activity. Animals would either be pretreated with vehicle or with different doses of a test compound and are dosed with vehicle or test compound three hours prior to injection of 0.05 mL of sterile 1% formalin under the dorsal skin of a hind paw. The number of paw flinches (responses) during the first phase (0-5 min.) and the second phase (20-35 min.) are scored and recorded. Flinch response can be compared with the mean score of a saline control group and calculated as percentage inhibition. The ED₅₀ is the dose of compound which produced 50% inhibition of nociceptive response in the first or second phase response.
% inhibition of nociceptive response can be calculated as:

$$100 \times \frac{\begin{matrix}( number of responses in vehicle group - \\ number of responses in compound group )\end{matrix}}{( number of responses in vehicle group )}$$

Student's t-test can be used for statistical analysis to determine the significance of compound effects.

Test C: Neuropathic Pain Model (Chronic Constriction Injury):

The anti-hyperalgesic properties of a compound may be tested with the Chronic Constriction Injury (“CCI”) model. The test is a model for neuropathic pain associated with nerve injuries that can arise directly from trauma and compression, or indirectly from a wide range of diseases such as infection, cancer, metabolic conditions, toxins, nutritional deficiencies, immunological dysfunction, and musculoskeletal changes. In the model a unilateral peripheral hyperalgesia is produced in rats by nerve ligation (G. J. Bennett, et al., Pain 33, 87-107 (1988)).

Procedurally, Sprague-Dawley rats (250-350 g) are anesthetized with sodium pentobarbital and the common sciatic nerve exposed at the level of the mid thigh by blunt dissection through the biceps femoris. A section of nerve (about 7 mm), proximal to the sciatic trifucation, is freed of tissue and ligated at four positions with chromic gut suture, with the suture tied with about 1 mm spacing between ligatures. The incision is closed in layers and the animals allowed to recuperate. Thermal hyperalgesia is measured using a paw-withdrawal test (K. Hargreaves, et al., Pain 32, 77-88 (1988)). To perform the test, animals are habituated on an elevated glass floor and a radiant heat source aimed at the mid-plantar hindpaw (sciatic nerve territory) through the glass floor with a 20 second cut-off to prevent injury to the skin. The latencies for the withdrawal reflex in both hind paws are recorded.

In this test, paws with ligated nerves show shorter paw withdrawal latencies compared to the unoperated or sham operated paws. Responses to test compounds are evaluated at different times after oral administration to determine the onset and duration of compound effect. When performing the test, groups of CCI rats would receive either vehicle or the test compound orally three times daily for 5 days. Paw withdrawal latencies can be measured each day 10 min. before and two or three hr. after the first daily dose. Compound efficacy is calculated as mean percentage decrease of hyperalgesia compared to a vehicle-treated group. Compound potencies may be expressed as the minimum effective dose (MED) in mg/Kg/day that yields a % decrease in hyperalgesia that is statistically significant, where the % anti-hyperalgesic effect may be calculated as follows:

$$\frac{( Mean of vehicle group - Mean of compound group )}{( Mean of vehicle group )} \times 100$$

Data analysis can be performed by the multiple means comparison (Dunnett's test).

Table 1 shows the results from Test A for the compounds of the invention.

TABLE 1

| | Test A |
|---|---|
| | Ki (nM) |
| Ex. 1 | 95.8 |
| Ex. 2 | 42.7 |
| Ex. 3 | 1150 |
| Ex. 4 | 449 |
| Ex. 5 | 146 |
| Ex. 6 | 268 |
| Ex. 7 | 173 |
| Ex. 8 | 124 |
| Ex. 9 | 159 |
| Ex. 10 | 91 |
| Ex. 11 | 227 |

1. A compound selected from:
7-chloro-4-hydroxy-2-(4-[1,3,4]oxadiazol-2-yl-benzyl)-1,2,5,10-tetrahydropyridazino[4,5-b]quinoline-1,10-dione;
7-chloro-4-hydroxy-2-[3-(1H-tetrazol-5-yl)-benzyl]-1,2,5,10-tetrahydropyridazino[4,5-b]quinoline-1,10-dione;
4-[(7-chloro-4-hydroxy-1,10-dioxo-2,5-dihydropyridazino[4,5-b]quinolin-2-yl)methyl]benzaldehyde;
N-(1-aza-2-{[4-[(7-chloro-4-hydroxy-1,10-dioxo(2,5-dihydropyridazino[4,5-b]quinolin-2-yl))methyl]phenyl}vinyl)(tert-butoxy)carboxamide;
2-({4-[2-aza-2-(2-pyridylamino)vinyl]phenyl}methyl)-7-chloro-4-hydroxy-2,5-dihydropyridazino[4,5-b]quinoline-1,10-dione;
N-(-1-aza-2-{4-[(7-chloro-4-hydroxy-1,10-dioxo(2,5-dihydropyridazino[4,5-b]quinolin-2-yl))methyl]phenyl}vinyl)benzamide, and
2-{[4-(2-aza-2-{[(2,4,6-trimethylphenyl)sulfonyl]amino}vinyl)phenyl]methyl}-7-chloro-4-hydroxy-2,5-dihydropyridazino[4,5-b]quinoline-1,10-dione.

2. A method for treating a subject suffering from pain comprising administering a pain-ameliorating effective amount of a compound selected from:
7-chloro-4-hydroxy-2-(4-[1,3,4]oxadiazol-2-yl-benzyl)-1,2,5,10-tetrahydropyridazino[4,5-b]quinoline-1,10-dione;
7-chloro-4-hydroxy-2-[3-(1H-tetrazol-5-yl)-benzyl]-1,2,5,10-tetrahydropyridazino[4,5-b]quinoline-1,10-dione;
7-chloro-4-hydroxy-2-[3-hydroxy-5(hydroxymethyl)-2-methyl(4-pyridyl)]methyl-1,2,5,10-tetrahydropyridazino[4,5-b]quinoline-1,10-dione;
7-chloro-4-hydroxy-2-(tetrahydrofuran-2-ylmethyl)-1,2,5,10-tetrahydropyridazino[4,5-b]quinoline-1,10-dione;
7-chloro-4-hydroxy-2-[5-(4-methoxyphenyl)-[1,3,4]oxadiazol-2-ylmethyl]-1,2,5,10-tetrahydropyridazino[4,5-b]quinoline-1,10-dione;
7-chloro-4-hydroxy-2-[1-(4-methoxycarbonylphenyl)ethyl)-1,2,5,10-tetrahydropyridazino[4,5-b]quinoline-1,10-dione;
4-[(7-chloro-4-hydroxy-1,10-dioxo-2,5-dihydropyridazino[4,5-b]quinolin-2-yl)methyl]benzaldehyde;
N-(1-aza-2-{[4-[(7-chloro-4-hydroxy-1,10-dioxo(2,5-dihydropyridazino[4,5-b]quinolin-2-yl))methyl]phenyl}vinyl)(tert-butoxy)carboxamide;
2-({4-[2-aza-2-(2-pyridylamino)vinyl]phenyl}methyl)-7-chloro-4-hydroxy-2,5-dihydropyridazino[4,5-b]quinoline-1,10-dione;
N-(-1-aza-2-{4-[(7-chloro-4-hydroxy-1,10-dioxo(2,5-dihydropyridazino[4,5-b]quinolin-2-yl))methyl]phenyl}vinyl)benzamide, and
2-{[4-(2-aza-2-{[(2,4,6-trimethylphenyl)sulfonyl]amino}vinyl)phenyl]methyl}-7-chloro-4-hydroxy-2,5-dihydropyridazino[4,5-b]quinoline-1,10-dione.

3. A pharmaceutical composition comprising a pain-ameliorating effective amount of a compound selected from:
7-chloro-4-hydroxy-2-(4-[1,3,4]oxadiazol-2-yl-benzyl)-1,2,5,10-tetrahydropyridazino[4,5-b]quinoline-1,10-dione;
7-chloro-4-hydroxy-2-[3-(1H-tetrazol-5-yl)-benzyl]-1,2,5,10-tetrahydropyridazino[4,5-b]quinoline-1,10-dione;
7-chloro-4-hydroxy-2-[3-hydroxy-5(hydroxymethyl)-2-methyl(4-pyridyl)]methyl-1,2,5,10-tetrahydropyridazino[4,5-b]quinoline-1,10-dione;
7-chloro-4-hydroxy-2-(tetrahydrofuran-2-ylmethyl)-1,2,5,10-tetrahydropyridazino[4,5-b]quinoline-1,10-dione;
7-chloro-4-hydroxy-2-[5-(4-methoxyphenyl)-[1,3,4]oxadiazol-2-ylmethyl]-1,2,5,10-tetrahydropyridazino[4,5-b]quinoline-1,10-dione;
7-chloro-4-hydroxy-2-[1-(4-methoxycarbonylphenyl)ethyl)-1,2,5,10-tetrahydropyridazino[4,5-b]quinoline-1,10-dione;
4-[(7-chloro-4-hydroxy-1,10-dioxo-2,5-dihydropyridazino[4,5-b]quinolin-2-yl)methyl]benzaldehyde;
N-(1-aza-2-{[4-[(7-chloro-4-hydroxy-1,10-dioxo(2,5-dihydropyridazino[4,5-b]quinolin-2-yl))methyl]phenyl}vinyl)(tert-butoxy)carboxamide;
2-({4-[2-aza-2-(2-pyridylamino)vinyl]phenyl}methyl)-7-chloro-4-hydroxy-2,5-dihydropyridazino[4,5-b]quinoline-1,10-dione;
N-(-1-aza-2-{4-[(7-chloro-4-hydroxy-1,10-dioxo(2,5-dihydropyridazino[4,5-b]quinolin-2-yl))methyl]phenyl}vinyl)benzamide, and
2-{[4-(2-aza-2-{[(2,4,6-trimethylphenyl)sulfonyl]amino}vinyl)phenyl]methyl}-7-chloro-4-hydroxy-2,5-dihydropyridazino[4,5-b]quinoline-1,10-dione;
together with a pharmaceutically-acceptable excipient or diluent;
